(19) United States
(12) Patent Application Publication
(10) Pub. No.: US 2025/0161506 A1
(43) Pub. Date: May 22, 2025

(54) Antibody Drug Conjugate Based on Metal N-Heterocyclic Carbene Complex, a Method of Preparing the Same and the Use of the Same in Anticancer Treatment

(71) Applicant: Laboratory for Synthetic Chemistry and Chemical Biology Limited, Hong Kong (HK)

(72) Inventors: Chi Ming CHE, Hong Kong (HK); Chun Ho CHU, Hong Kong (HK)

(21) Appl. No.: 18/925,062

(22) Filed: Oct. 24, 2024

Related U.S. Application Data
(60) Provisional application No. 63/601,224, filed on Nov. 21, 2023.

(51) Int. Cl. A61K 51/10; C07B 59/00

(52) U.S. Cl. CPC A61K 51/1051; C07B 59/008; A61K 2121/00; C07B 2200/05

(57) ABSTRACT

The present invention provides an antibody drug conjugate composed of an anti-HER2 trastuzumab monoclonal antibody and an iridium(III) N-heterocyclic carbene (NHC) complex connected with a cathepsin B-cleavable GGFG tetrapeptide linker, a method of preparing the same and a method of using the same for anticancer treatment. The provided antibody drug conjugate has improved tumor target specificity and delivery.

### CROSS-REFERENCE TO RELATED APPLICATIONS

The present application claims priority from the U.S. Provisional Patent Application No. 63/601,224 filed 21 Nov. 2023, and the disclosure of which is incorporated herein by reference in its entirety.

### FIELD OF THE INVENTION

The present invention generally relates to antibody-drug conjugate (ADC) and specifically relates to an ADC based on metal N-heterocyclic carbene (NHC) complexes.

### BACKGROUND OF THE INVENTION

Cancer is the leading cause of death worldwide. However, due to the nonselective mechanism of action, the clinically used chemo-drugs exhibit limited selectivity against cancer cells and thus cause serious side effects. Much efforts have been devoted to develop new anticancer drugs that can be specifically delivered to the tumor sites. ADC has attracted much attention for the research and development of anticancer drugs. It is composed of a monoclonal antibody that targets a tumor-specific antigen, which is then covalently conjugated with a cytotoxic drug via a chemical linker, thereby combining both the advantages of highly specific targeting ability of antibodies and highly potent drugs. To date, 14 ADCs have been approved by FDA, of which the cytotoxic payloads mainly include potent tubulin inhibitors and DNA damaging agents. While the former is less effective against static cancer cells, the latter is too toxic causing serious adverse effects due to the unavoidable off-site toxicity of ADC in the healthy non-cancerous cells. Therefore, there is still a strong need to develop novel ADC payloads ideally with better therapeutic indexes. On the other hand, metal complexes have unique three-dimensional scaffolds for binding to drug target proteins that differ from the conventional small molecular drugs. Remarkable advances have been achieved in the development of metal N-heterocyclic carbene (NHC) complexes as anticancer agents for several decades. The strong σ-donor properties of NHC results in stable and strong metal-NHC bonds that are resistant to physiological reductants and nucleophiles. In addition to the chemical and thermal stability, the NHC ligands can be modified easily to endow additional functionalities to the complexes, making them ideal and promising candidates of metallodrugs, and hence novel payloads of ADC.

However, the conjugation site of the drug to antibody could have significant impact on the potency of the drug, which requires careful consideration.

### SUMMARY OF THE INVENTION

According to a first aspect of the present invention, an antibody drug conjugate (ADC) composed of a tumor-targeting antibody, selected from but not limiting to anti-HER2 trastuzumab, anti-EGFR cetuximab, anti-TROP2 sacituzumab, anti-nectin-4 enfortumab, anti-tissue factor tisotumab and anti-FRα mirvetuximab, and a metal N-heterocyclic carbene (NHC) complex covalently connected with a linker, selected from but not limiting to cathepsin B-cleavable GGFG tetrapeptide linker, acid-cleavable carbonate linker, and glutathione-cleavable disulfide linker, is provided with improved tumor target specificity and delivery.

According to a second aspect of the present invention, a method of preparing the ADC of the first aspect of the present invention is provided. The method comprises: reacting a Fmoc-protected GGFG peptide acetate with a hydroxyl-functionalized iridium (III)N-heterocyclic carbene (NHC) complex to form a first product; deprotecting Fmoc from the first product with 20% piperidine in DMF to form a second product and condensing the second product with 6-maleimidocaproic acid N-succinimidyl ester to obtain a maleimide drug precursor; and conjugating the maleimide drug precursor with a partially reduced trastuzumab to form the ADC.

The resulting ADC can cause a significant 10-fold increase in drug accumulation in tumor and a 90% reduction in iridium levels in the kidney compared with the treatment of the parent iridium complex. It is worth noting that the conjugation site of the drug to antibody could largely affect the potency of the drug, which requires careful consideration. The present invention successfully demonstrated that the modification of iridium (III) pyridinium-NHC complex with a hydroxyl group on the pyridinium ring for bioconjugation did not significantly affect the half-maximal inhibitory concentration (IC₅₀) values, which provides a useful handle for conjugation.

### BRIEF DESCRIPTION OF THE DRAWINGS

Embodiments of the invention are described in more details hereinafter with reference to the drawings, in which:

FIG. 1 shows a synthetic route of the pyridinium triflate derivative and the two hydroxyl analogues of Ir(III)-NHC complexes according to one embodiment of the present invention;

FIG. 2 shows comparison of effects on the IC₅₀ values of the functionalized complexes after different modification stages with respect to the parent complex;

FIG. 3 shows an ADC comprising of anti-HER2 trastuzumab monoclonal antibody and Ir(III)-NHC moiety (ADC-Ir) according to one embodiment of the present invention;

FIGS. 4A and 4B shows the synthetic route for the preparation of an ADC according to one embodiment of the present invention;

FIG. 5 shows sodium dodecyl-sulfate polyacrylamide gel electrophoresis (SDS-PAGE) analysis of the ADC;

FIG. 6 shows the absorption spectrum of ADC in the UV and visible regions;

FIG. 7 shows mass spectrometry analysis on the ADC; and

FIGS. 8A and 8B show in vivo bio-distribution of iridium content in SKOV-3 tumor-bearing nude mice due to administration of ADC-Ir after 1 day and 7 days, respectively.

### DETAILED DESCRIPTION

In the following description, exemplary embodiments of the present invention are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

According to one aspect of the present invention, an antibody drug conjugate is composed of an anti-HER2 trastuzumab monoclonal antibody and a metal N-heterocyclic carbene (NHC) complex connected with a maleimide drug precursor.

In some embodiments, the metal N-heterocyclic carbene (NHC) complex may be an iridium (III)N-heterocyclic carbene (NHC) complex with the formula:

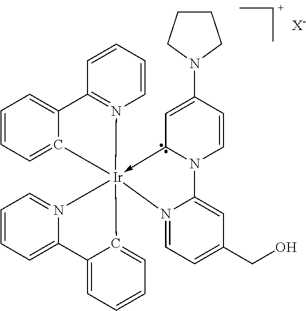

wherein X is a counter anion selected from CF₃SO₃, PF₆, Cl, Br or I.

The maleimide drug precursor may be a cathepsin B-cleavable GGFG tetrapeptide linker having a formula:

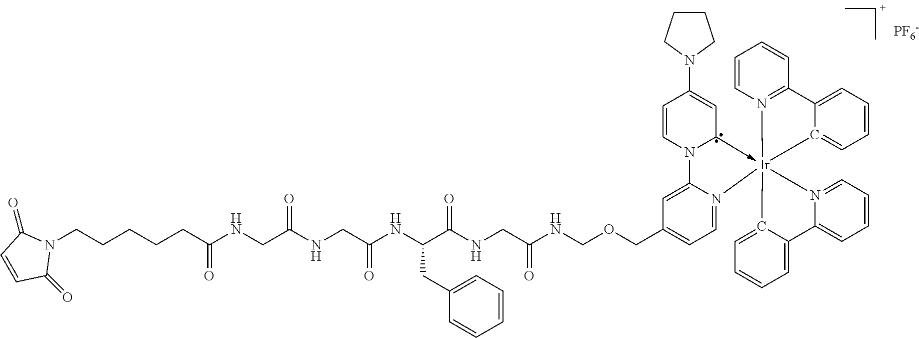

The metal center of NHC complex may be iridium, iron, osmium, ruthenium, and rhodium.

The 2-phenylpyridine and [1,2′-bipyridin]-1-ium ligand of NHC complex may be derivatized with, including but not limiting to, halogen, hydroxyl, alkyl, aryl, acyl, alkoxy, acyloxy, amino, nitro, acylamino, aralkyl, cyano, carboxyl, thio, styryl, aminocarbonyl, carbamoyl, aryloxycarbonyl, phenoxycarbonyl, or an alkoxycarbonyl group.

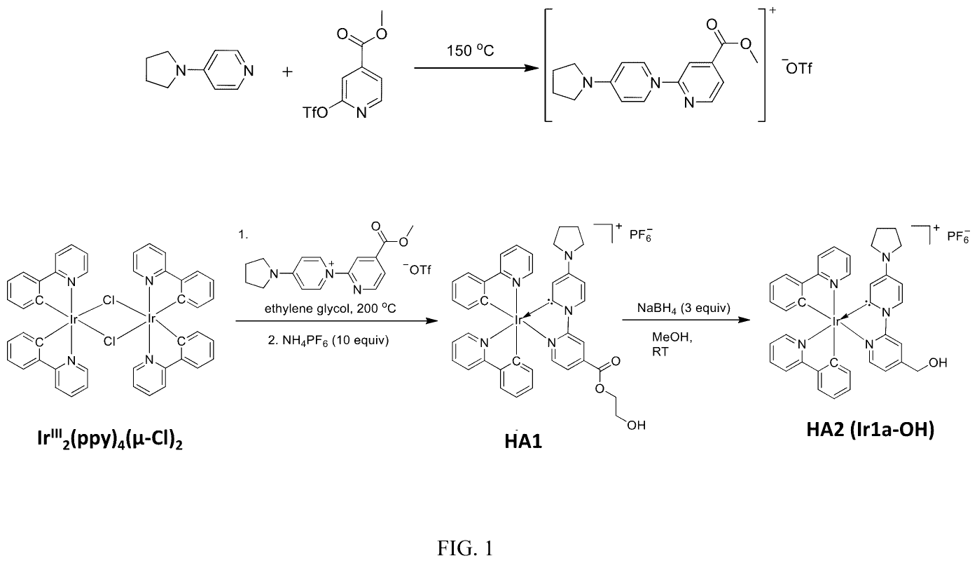

To functionalize metal complexes for bioconjugation, a hydroxyl group was introduced to the metal ligand with different structures. FIG. 1 shows a synthetic route of the pyridinium triflate derivative and the two hydroxyl analogues (HAs) of Ir(III)-NHC complexes. The pyridinium triflate derivative was prepared by heating the 4-pyrrolidinylpyridine with methyl 2-(((trifluoromethyl) sulfonyl)oxy) isonicotinate at 150° C. under argon. The μ-chloro-bridged dimer was reacted with the pyridinium triflate in ethylene glycol under reflux to afford hydroxyl analog 1 (HA1), which was then underwent reduction with sodium borohydride to give hydroxyl analog 2 (HA2).

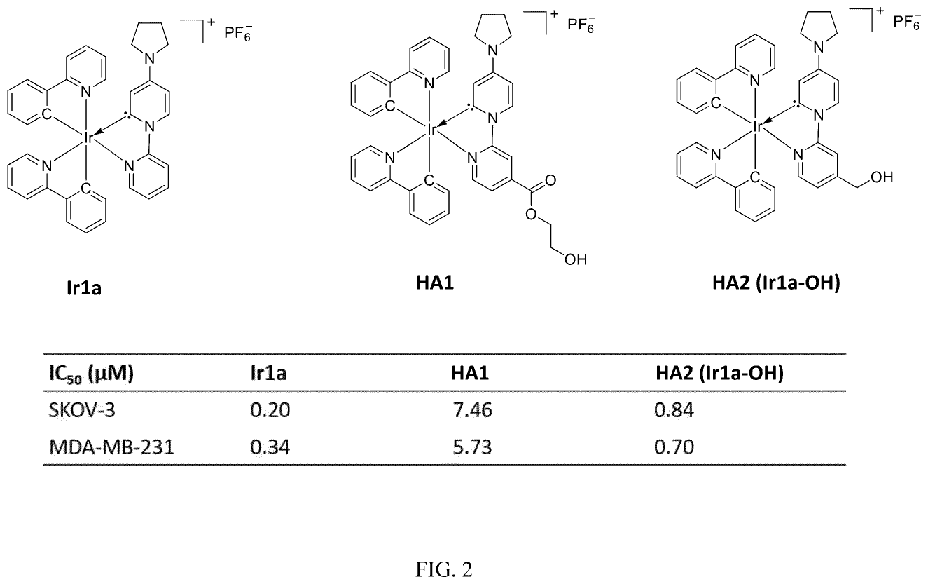

The effect on the IC₅₀ values of the functionalized complex after different modification stages (HA1 and HA2) are examined and compared to the parent complex. Referring to FIG. 2, interestingly, the potency of the complex functionalized with HA1 dropped significantly with the IC₅₀ values ranging from 5.73 to 7.46 μM, compared to Ir1a (IC_{50=0.20} to 0.34 μM). However, another HA2 (Ir1a-OH) without the ester group exhibited similar IC₅₀ values as those of Ir1a. This result indicated that change in the structure of metal ligand could largely affect the potency of the drug.

After optimizing the chemical structure for bioconjugation, the submicromolar anticancer potency and availability of functionalization sites of Ir1a render it to be a promising cytotoxic payload for ADCs.

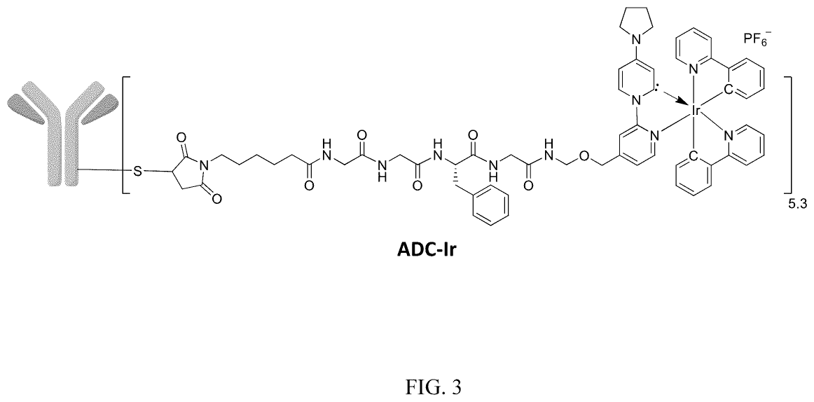

FIG. 3 shows an ADC comprising of anti-HER2 trastuzumab monoclonal antibody and Ir1a moiety (ADC-Ir) according to one embodiment of the present invention. In some embodiments, the ADC is prepared by conjugating a Ir1a-OH with trastuzumab via a cathepsin B-cleavable GGFG tetrapeptide linker with reference to the design of the FDA-approved trastuzumab deruxtecan. The drug-to-antibody (DAR) ratio was determined to be 5.3 by mass spectrometry. In other embodiments, the antibody is selected from but not limiting to those target the overexpressed EGFR, TROP2, nectin-4, tissue factor, or FRα on cancer cells. In other embodiments, the linker connecting antibody and payload can be comprised of any acid-labile, glutathione-labile, and enzyme-cleavable unit.

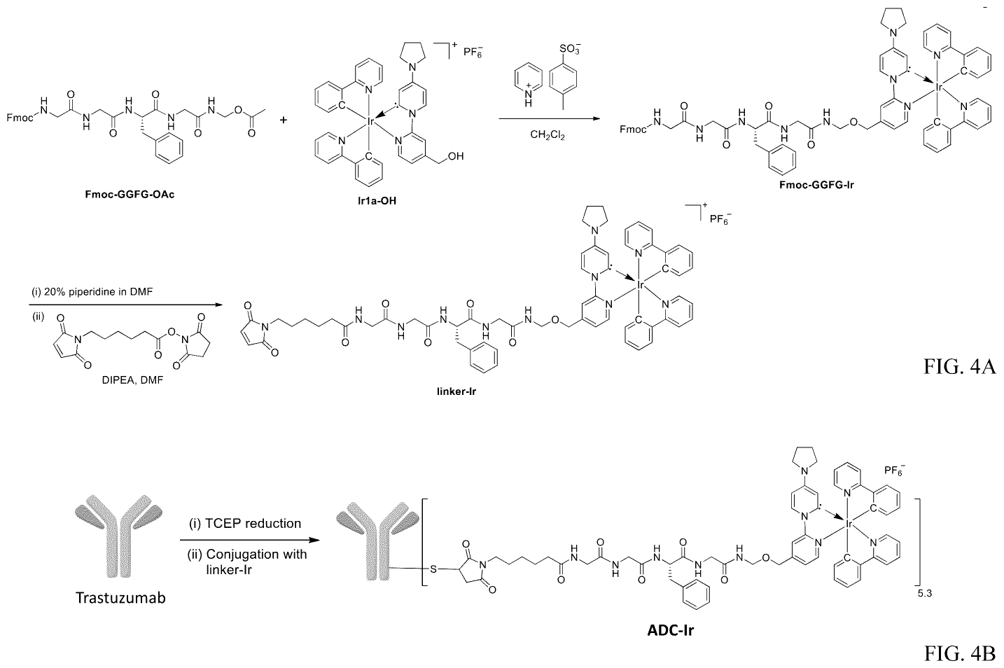

FIGS. 4A and 4B shows the synthetic route for the preparation of the ADC. Referring to FIG. 4A, the Fmoc-protected peptide acetate (Fmoc-GGFG-OAc) reacted with iridium hydroxyl (Ir1a-OH) produce a first product (Fmoc-GGFG-Ir), followed by Fmoc deprotection with 20% piperidine in DMF to form a second product and subsequent condensation of the second product with 6-maleimidocaproic acid N-succinimidyl ester in the presence of DIPEA in DMF to afford the maleimide drug precursor (i.e. linker-Ir).

Referring to FIG. 4B, the four interchain disulfide bonds of antibody trastuzumab, after reduction with tris(2-carboxyethyl) phosphine hydrochloride, could conjugate with linker-Ir via Michael addition to produce ADC-Ir. After removal of the unreacted linker-Ir by Zeba spin desalting column (7 kDa MWCO), the ADC-Ir was obtained. The protein concentration was determined by the standard bicinchoninic acid (BCA) assay and the yield was determined to be 85%.

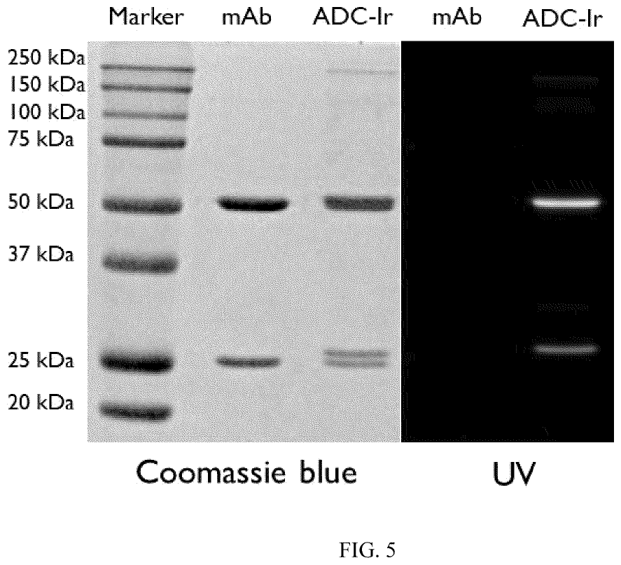

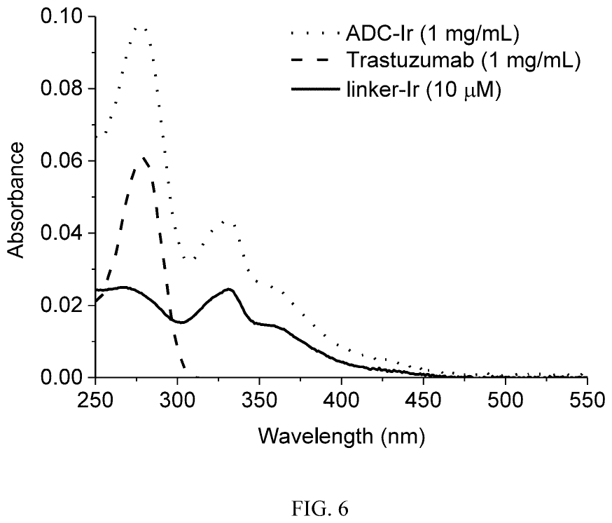

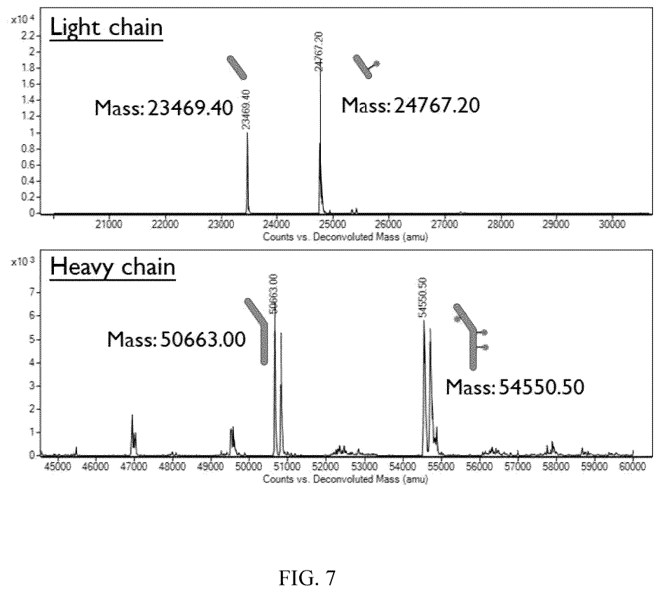

As shown in FIG. 5, SDS-PAGE analysis of ADC indicated that both the light chain and heavy chain fragments of trastuzumab (mAb) migrated at a larger kDa after conjugation with metal complexes, which could also be observed in UV light. The UV-Vis absorption spectrum of ADC showed the absorption peaks of both Ir1a and trastuzumab (FIG. 6). Further analysis by mass spectrometry suggested that the drug-to-antibody ratio (DAR) was determined as 5.3 (FIG. 7).

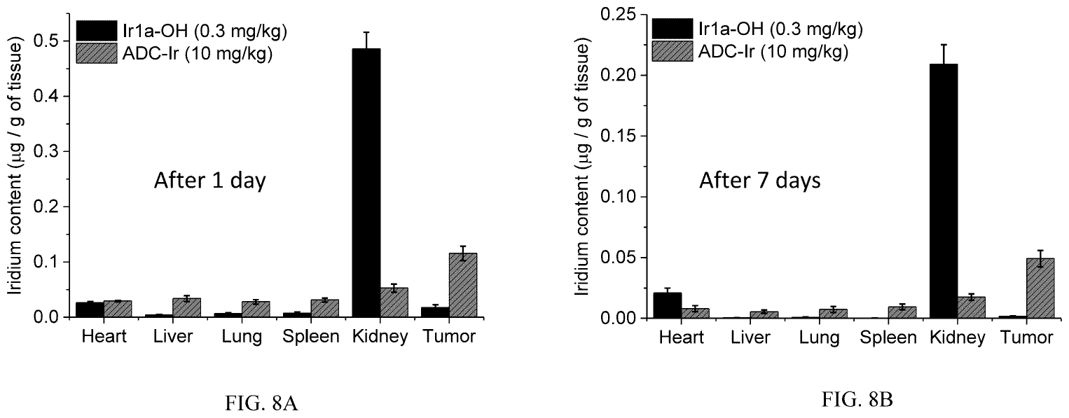

To examine the in vivo tumor targeting effect, ADC-Ir(10 mg/kg) or Ir1a-OH (0.3 mg/kg), both with equivalent content of Ir1a moieties, was injected intravenously to SKOV-3 tumor-bearing nude mice. In vivo bio-distribution of iridium content was analyzed by ICP-MS after 1 day and 7 days. As shown in FIGS. 8A and 8B, administration of ADC-Ir resulted in a significant 10-fold increase in iridium accumulation in tumors and a 90% reduction in iridium levels in the kidney compared with the effect of Ir1a-OH treatment. These data demonstrate that Ir1a is suitable for use in ADC-based strategies, showing increased tumor delivery in vivo and reduced systemic payload distribution in normal tissues.

### Synthesis of Hydroxyl-Functionalized Iridium (III) NHC Complexes

A pyridinium triflate was prepared using methyl 2-(((trifluoromethyl) sulfonyl)oxy) isonicotinate and heating with 4-pyrrolidinylpyridine at 150° C. ¹H NMR (600 MHZ, CDCl3, 298 K): δ 8.86 (d, J=7.8 Hz, 2H), 8.71 (d, J=4.8 Hz, 1H), 8.35 (s, 1H), 8.00 (d, J=4.8 Hz, 1H), 7.06 (d, J=7.8 Hz, 2H), 4.02 (s, 3H), 3.71 (m, 4H), 2.32 (m, 4H).

Referring back to FIG. 1. To an ethylene glycol solution (30 mL×1) of pyridinium triflate derivative (120 mg, 0.277 mmol), a suspension of μ-chloro-bridged iridium dimer Ir^{III}₂(ppy)₄(μ-Cl)₂ (200 mg, 0.186 mmol) was added. The mixture was degassed in vacuo for 15 min. After that, the mixture was heated at 200° C. for 12 h. The color of the mixture slowly changed into orange. After 12 h, ammonium hexafluorophosphate NH₄PF₆ (10 equiv.) was added to give orange precipitates (HA1). The precipitates are collected and redissolved in methanol (10 mL×1). Sodium borohydride NaBH₄ (3 equiv.) was added and the resulting mixture was stirred for 6 h. The color of the mixture changed into yellow. After 6 h, the solvent was removed in vacuo and the crude compound was extracted with dichloromethane (10 mL×3). The crude product was then further purified by column chromatography (SiO₂, DCM/EA, v/v=10:1) to give HA2. (31 mg, yield: 37%).

¹H NMR (600 MHZ, CD₃CN, 298 K): δ 8.59 (d, J=7.8 Hz, 1H), 8.12 (d, J=7.8 Hz, 1H), 8.05 (d, J=7.8 Hz, 1H), 7.94 (s, 1H), 7.88 (t, J=4.8 Hz, 1H), 7.76-7.83 (m, 3H), 7.71 (d, J=4.8 Hz, 1H), 7.63 (d, J=5.4 Hz, 1H), 7.51 (d, J=5.4 Hz, 1H), 7.21 (d, J=5.4 Hz, 1H), 7.14 (t, J=6.0 Hz, 1H), 7.07 (t, J=6.0 Hz, 1H), 6.95-6.99 (m, 2H), 6.89 (t, J=7.2 Hz, 1H), 6.83 (t, J=7.2 Hz, ¹H), 6.75 (d, J=7.2 Hz, 1H), 6.66 (d, J=7.2 Hz, 1H), 6.55 (dd, J1=8.4 Hz, J2=3.0 Hz, 1H), 5.91 (d, J=3.0 Hz, 1H), 4.73 (s, 2H), 3.74 (br, 1H, OH), 3.40-3.47 (m, 2H), 2.76-2.96 (m, 2H), 1.82-1.90 (m, 4H).

¹³C {¹H} NMR (150 MHz, CD₃CN, 298 K): δ 177.03, 166.50, 166.35, 157.37, 157.23, 156.84, 156.60, 151.64, 149.39, 148.48, 148.03, 145.29, 144.64, 139.35, 139.01, 136.98, 136.85, 136.22, 131.35, 130.92, 125.59, 125.49, 124.28, 124.10, 122.62, 122.12, 120.98, 120.54, 115.14, 110.45, 105.78, 62.85, 48.81, 48.24, 25.60, 25.51.

¹⁹F {¹H} NMR (376 MHz, CD₃CN, 298 K): δ-73.1

³¹P {¹H} NMR (162 MHZ, CD₃CN, 298 K): δ-135.1

HR-ESI-MS: m/z for C₃₇H₃₃IrN₅O⁺ [M-PF₆]⁺ calcd: 756.2314; found: 756.2328.

### Synthesis of Linker-Ir

A Fmoc-protected peptide acetate (Fmoc-GGFG-OAc) (4.0 mg, 6.2 μmol) was reacted with a hydroxyl-functionalized iridium (III)N-heterocyclic carbene (NHC) complex (Ir1a-OH) (4.0 mg, 4.4 μmol) in the presence of pyridinium p-toluenesulfonate (0.12 mg, 0.44 μmol) in dichloromethane CH₂Cl₂. The reaction mixture was refluxed at 40° C. overnight and the solvent was removed in vacuo. The crude product was purified by semi-preparative High Performance Liquid Chromatography (HPLC) to afford Fmoc-GGFG-Ir(2.2 mg, 31%) after lyophilization.

Subsequently, Fmoc was deprotected from the Fmoc-GGFG-Ir with 20% piperidine in DMF and condensing the resulting product with 6-maleimidocaproic acid N-succinimidyl ester in DIPEA in DMF to obtain a maleimide drug precursor (linker-Ir).

¹H NMR (600 MHZ, DMSO-d₆): δ 8.97 (d, J=7.9 Hz, 1H), 8.74 (d, J=4.5 Hz, 1H), 8.48 (s, 1H), 8.43 (d, J=5.6 Hz, 1H), 8.27-8.29 (m, 3H), 8.20 (d, J=8.1 Hz, 1H), 8.12-8.13 (m, 1H), 7.95 (t, J=6.3 Hz, 1H), 7.83-7.89 (m, 5H), 7.72 (d, J=5.4 Hz, 1H), 7.65-7.67 (m, 2H), 7.62 (t, J=5.9 Hz, 1H), 7.58 (dd, J1=5.7 Hz, J2=1.8 Hz, 1H), 7.37-7.41 (m, 3H), 7.28-7.32 (m, 3H), 7.14-7.25 (m, 6H), 6.91-6.95 (m, 2H), 6.85 (t, J=7.6 Hz, 1H), 6.80 (t, J=7.6 Hz, 1H), 6.60-6.66 (m, 3H), 5.79 (t, J=3.2 Hz, 1H), 4.69 (d, J=6.5 Hz, 2H), 4.64 (s, 2H), 4.41-4.46 (m, 1H), 4.18-4.25 (m, 3H), 3.57-3.78 (m, 8H), 2.98-3.03 (m, 1H), 2.85-2.88 (m, 1H), 2.75-2.79 (m, 1H), 2.61-2.67 (m, 1H), 1.75-1.88 (m, 4H).

¹³C NMR (150 MHz, DMSO-d₆): δ 175.96, 171.99, 170.74, 170.04, 165.69, 165.39, 165.29, 157.05, 156.35, 156.02, 153.08, 150.45, 148.50, 147.35, 147.14, 144.48, 144.26, 143.79, 143.03, 141.17, 139.88, 139.05, 138.64, 138.32, 137.89, 136.92, 136.05, 135.15, 130.75, 130.20, 129.55, 129.40, 128.53, 128.10, 127.77, 127.53, 126.72, 125.69, 125.22, 125.01, 124.16, 123.86, 122.57, 121.93, 121.86, 121.76, 120.57, 120.50, 120.17, 114.08, 110.87, 110.25, 105.53, 69.97, 67.66, 66.23, 48.15, 47.40, 47.06, 45.11, 43.99, 42.75, 42.41, 37.58, 24.90, 24.79.

HR-ESI-MS: m/z for C₆₃H₆₅IrN₁₁O₈⁺ [M-PF₆]⁺ calcd: 1296.4647; found: 1296.4689.

Reverse-phase HPLC separation was performed on a XBridge BEH300 Prep C18 column (5 μm, 10 mm×250 mm) at a flow rate of 3 mL/min using an Agilent 1260 Infinity II LC system equipped with an Agilent 1260 Quat Pump and an Agilent 1260 DAD WR. The condition used for the analysis was set as follows: solvent A=0.1% formic acid (FA) in deionized water and solvent B=0.1% FA in acetonitrile; gradient: 95% A+5% B in the first 5 min, then changed to 85% A+15% B in 10 min, further changed to 5% A+95% B in 30 min, maintained under this condition for 5 min, changed to 95% A+5% B in 5 min, maintained under this condition for further 5 min.

### In Vitro Anti-proliferative Activity (NBB Assay)

SKOV-3 ovarian cancer cells (5×10³ cells/well) in DMEM were seeded in a microtiter plate (96 well) and incubated overnight before the treatment. Different concentrations of Ir1a and Ir1a-OH and the vehicle control (0.5% DMSO) were added into the wells and then incubated for 72 h. After the media was removed, the cells were fixed with formaldehyde (3%; 50 μL) in PBS and stained with NBB reagent (0.05%, 0.1 M sodium acetate, 9% acetic acid; 50 μL) for overnight. The stained cells in each well were washed thrice gently with deionized water and solubilized in sodium hydroxide solution (50 mM; 100 μL). The cell viability was determined by measuring the absorbance of 620 nm in each well by microplate reader. The experiments were repeated in triplicate. The IC₅₀ values are presented as the mean±standard deviation.

### Preparation and Characterization of ADC-Ir

Trastuzumab (MCE, 2.5 mg/mL) was dissolved in borate buffer (pH 8.0) and TCEP (10 equiv.) was added to the reaction mixture for 1.5 h. After the reduction of disulphide bond, linker-Ir(16 equiv.) dissolved in DMSO (2% v/v) was added and the reaction mixture was stirred for 2 h. The ADC-Ir conjugate was purified by using Zeba™ Spin Desalting Column (Thermo Fisher, 7K MWCO) according to the manufacturer's protocol. The protein concentration was determined by the standard bicinchoninic acid (BCA) assay and the yield was determined to be 85%. The conjugate was analyzed by Thermo Scientific μDrop and reducing SDS-PAGE with 12% acrylamide with 4% stacking gel as standard.

The antibody and the conjugate were analyzed on a MAbPac Reversed Phase HPLC Column (4 μm, 3 mm×50 mm) at a flow rate of 0.3 mL/min. Electrospray ionization (ESI) mass spectra were recorded on an Agilent 6546 LC/Q-TOF mass spectrometer. The condition used for the analysis was set as follows: solvent A=0.1% FA in deionized water and solvent B=0.1% FA in acetonitrile; gradient: 80% A+20% B in the first 0.5 min, then changed to 40% A+60% B in 3 min, maintained under this condition for 5 min, further changed to 80% A+20% B in 0.5 min, maintained under this condition for 1 min.

The average drug-to-antibody (DAR) ratio was determined by using the corresponding peak areas of the chromatograms in the deconvoluted mass spectra, which was found to be 5.3.

### In Vivo Biodistribution Assay

All animal experiments were conducted under the guidelines approved by the Committee on the Use of Live Animals in Teaching and Research of the University of Hong Kong. Female BALB/cAnN-nu (nude) mice (5-8 weeks old) were housed in the Laboratory Animal Unit, The University of Hong Kong. The mice were freely access to food and water. SKOV-3 ovarian cancer cells (1×10⁷) in PBS (100 μL) were injected into the right flanks of each mouse subcutaneously. Once the tumor volumes reached about 50 mm³, the mice were divided into 3 groups (n=6 for each group): (A) PBS control, (B) ADC-Ir(10 mg/kg) and (C) Ir1a-OH (0.3 mg/kg). After the intravenous administration, the mice were sacrificed at 1-day and 7-day post-injection. The tumours and major organs were collected and digested with nitric acid overnight at 70° C., followed by the ICP-MS (Agilent Technologies 7500A) analysis to quantify the iridium content.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. An antibody drug conjugate (ADC) composed of a tumor-targeting antibody connected with a metal N-heterocyclic carbene (NHC) complex with a linker;
wherein the linker is non-cleavable or cleavable upon stimulation by acid, glutathione or enzyme.

2. The ADC of claim 1, wherein the tumor-targeting antibody targets a tumor-associated receptor selected from HER2, TROP2, Nectin4, folate receptor alpha, or EGFR.

3. The ADC of claim 1, wherein the tumor-targeting antibody is an anti-HER2 trastuzumab.

4. The ADC of claim 1, wherein the metal N-heterocyclic carbene (NHC) complex is an iridium (III)N-heterocyclic carbene (NHC) complex with a 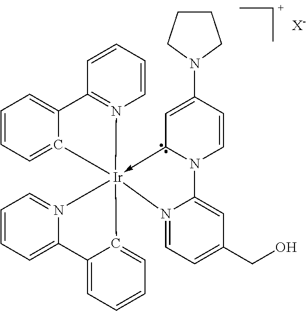
wherein X is a counter anion selected from CF₃SO₃, PF₆, Cl, Br or I.

5. The ADC of claim 1, wherein the linker is a cathepsin B-cleavable GGFG tetrapeptide linker.

6. A method of preparing an antibody drug conjugate of claim 1, comprising:
reacting a Fmoc-protected GGFG peptide acetate with a hydroxyl-functionalized iridium (III)N-heterocyclic carbene (NHC) complex to form a first product;
deprotecting Fmoc from the first product with 20% piperidine in DMF to form a second product and condensing the second product with 6-maleimidocaproic acid N-succinimidyl ester to obtain an iridium (III) NHC complex-loaded cathepsin B-cleavable GGFG tetrapeptide linker with the following formula: 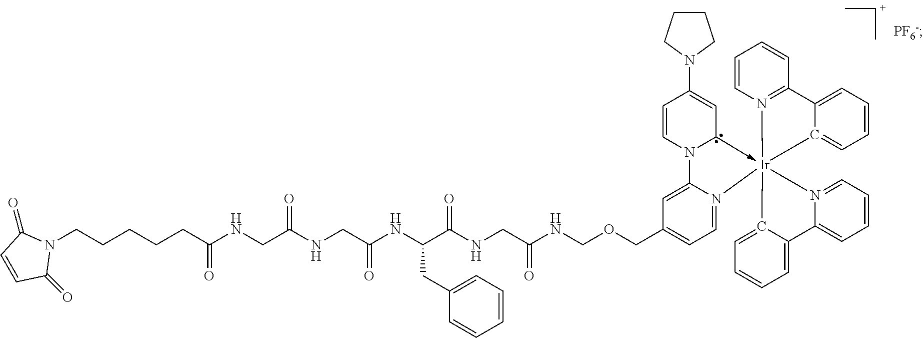
and
conjugating the iridium (III) NHC complex-loaded cathepsin B-cleavable GGFG tetrapeptide linker with a reduced trastuzumab to form the antibody drug conjugate.

7. The method according to claim 6, wherein the hydroxyl-functionalized iridium (III) NHC complex is prepared by:
reacting a μ-chloro-bridged iridium dimer with a pyridinium triflate in ethylene glycol solution under reflux to obtain a first mixture;
precipitating the first mixture with ammonium hexafluorophosphate to obtain a first product;
dissolving the first product in methanol; and
reducing the dissolved precipitates with sodium borohydride to form a second product; and
extracting the hydroxyl-functionalized iridium (III) NHC complex from the second product with dichloromethane.

8. The method according to claim 7, wherein the pyridinium triflate derivative is prepared by heating 4-pyrrolidinylpyridine with methyl 2-(((trifluoromethyl) sulfonyl)oxy) isonicotinate at 150° C.

9. The method according to claim 6, wherein the iridium (III) NHC complex-loaded cathepsin B-cleavable GGFG tetrapeptide linker is conjugated with the reduced trastuzumab via Michael addition.

10. The method according to claim 6, wherein the reduced trastuzumab is prepared by reducing four interchain disulfide bonds of a trastuzumab with tris(2-carboxyethyl) phosphine hydrochloride.

11. A method of using the antibody drug conjugate of claim 1 in anticancer treatment.
